(19) United States
(12) Patent Application Publication
(10) Pub. No.: US 2026/0041798 A1
(43) Pub. Date: Feb. 12, 2026

(54) RADIOPAQUE HYDROLYSABLE HYDROGELS AND METHODS OF TREATMENT USING SAME

(71) Applicant: Boston Scientific Scimed, Inc., Maple Grove, MN (US)

(72) Inventors: Yen-Hao Hsu, Shrewsbury, MA (US); Joseph Thomas Delaney, Jr., Minneapolis, MN (US)

(73) Assignee: Boston Scientific Scimed, Inc., Maple Grove, MN (US)

(21) Appl. No.: 19/296,077

(22) Filed: Aug. 11, 2025

Related U.S. Application Data
(60) Provisional application No. 63/682,178, filed on Aug. 12, 2024.

(51) Int. Cl. A61K 49/04

(52) U.S. Cl. CPC A61K 49/0442; A61K 49/0457; A61K 2123/00

(57) ABSTRACT

In some aspects, the present disclosure provides a system for forming a hydrogel that comprises: a reactive multi-arm polymer that comprises three or more polymer arms linked to a core region, at least three of the three or more polymer arms comprising a cyclic-imidyl ester group, a hydrolysable ester group, an iodinated lactone residue, an amide group and a hydrophilic polymer segment, where the cyclic-imidyl ester group is linked to the iodinated lactone residue through the hydrolysable ester group, the iodinated lactone residue is linked to the hydrophilic polymer segment through the amide group, and the hydrophilic polymer segment is linked to the core region; and a polyamino compound comprising at least two amino (—NH₂) groups, wherein the reactive multi-arm polymer and the polyamino compound react to form a crosslinked hydrogel. Other aspects pertain to crosslinked hydrogels formed from such systems and methods of treatment using such systems.

### CROSS-REFERENCE TO RELATED APPLICATIONS

This application claims the benefit of U.S. Provisional Patent Application Ser. No. 63/682,178 filed on Aug. 12, 2024, the disclosure of which is incorporated herein by reference.

### FIELD

The present disclosure relates to radiopaque hydrolysable hydrogels, to crosslinkable systems for forming such radiopaque hydrolysable hydrogels, and to methods of treatment using such radiopaque hydrolysable hydrogels.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion of the succinimidyl glutarate end groups have been replaced with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of about 6-9 months. The breakdown occurs primarily through the hydrolysis of the ester linkages in the glutarate groups.

The present disclosure provides implantable hydrogel alternatives to SpaceOAR Vue®, thereby expanding the range of medical applications for the hydrogels.

### SUMMARY

In some aspects, the present disclosure provides a system for forming a hydrogel that comprises (a) a reactive multi-arm polymer that comprises three or more polymer arms linked to a core region, at least three of the three or more polymer arms comprising a cyclic-imidyl ester group, a hydrolysable ester group, an iodinated lactone residue, an amide group and a hydrophilic polymer segment, where the cyclic-imidyl ester group is linked to the iodinated lactone residue through the hydrolysable ester group, the iodinated lactone residue is linked to the hydrophilic polymer segment through the amide group, and the hydrophilic polymer segment is linked to the core region; and (b) a polyamino compound comprising at least two amino (—NH₂) groups, wherein the reactive multi-arm polymer and the polyamino compound react to form a crosslinked hydrogel.

In some embodiments, the hydrophilic polymer segment is selected from a polyalkylene oxide segment, a polyester segment, a polyoxazoline segment, a polydioxanone segment, a polypeptide segment, an acrylic segment, an acrylamide segment, and a polysaccharide segment.

In some embodiments, which can be used in conjunction with the above aspects and embodiments, the iodinated lactone residue comprises at least one iodinated aromatic group.

In some embodiments, which can be used in conjunction with the above aspects and embodiments, the iodinated lactone residue is an iodinated benzolactone residue. In some of these embodiments, the iodinated benzolactone residue is selected from an iodinated 1-benzofuran-2-one compound reside, an iodinated 2-benzofuran-1-one compound reside, an iodinated chroman-2-one compound reside, and an iodinated 1-benzoxepin-2-one compound reside.

In some embodiments, which can be used in conjunction with the above aspects and embodiments, the at least three polymer arms comprise a cyclic-imidyl-oxycarbonyl-C₂-C₈-alkylene-carbonyloxy-group linked to the iodinated lactone residue.

In some embodiments, which can be used in conjunction with the above aspects and embodiments, the at least three polymer arms comprise a cyclic-imidyl-oxycarbonyl-1,2-dimethylene-carbonyloxy-end group, a cyclic-imidyl-oxycarbonyl-1,3-trimethylene-carbonyloxy-end group, a cyclic-imidyl-oxycarbonyl-1,4-tetramethylene-carbonyloxy-end group, a cyclic-imidyl-oxycarbonyl-1,5-pentamethylene-carbonyloxy-end group, or a cyclic-imidyl-oxycarbonyl-1,6-hexamethylene-carbonyloxy-end group.

In some embodiments, which can be used in conjunction with the above aspects and embodiments, the reactive multi-arm polymer is formed by a process that comprises (a) reacting a multi-arm polymer that comprises three or more polymer arms linked to the core region, at least three of the polymer arms comprising the hydrophilic polymer segment and an amino end group, with an iodinated lactone in a ring-opening reaction to form at least three polymer arms that comprise a hydroxy-containing iodinated lactone residue that is linked to the hydrophilic polymer segment through a linkage that comprises the amide group; (b) reacting the reaction product of step (a) with a cyclic anhydride in a ring opening reaction to form at least three polymer arms that comprise carboxyl end groups that a linked to the iodinated lactone residue through a linkage that comprises an ester group; and (c) reacting the reaction product of step (b) with an N-hydroxy cyclic imide compound in an amide coupling reaction to form the reactive multi-arm polymer.

In some embodiments, which can be used in conjunction with the above aspects, the cyclic anhydride has a ring that comprises a —C₂-C₈-alkylene-group, and the at least three polymer arms comprise a cyclic-imidyl-oxycarbonyl-C₂-C₈-alkylene-carbonyloxy-group.

In some embodiments, which can be used in conjunction with the above aspects and embodiments, the iodinated lactone comprises at least one iodinated aromatic group.

In some embodiments, which can be used in conjunction with the above aspects and embodiments, the iodinated lactone is an iodinated benzolactone. In some of these embodiments, the iodinated benzolactone is selected from an iodinated 1-benzofuran-2-one compound, an iodinated 2-benzofuran-1-one compound, an iodinated chroman-2-one compound, and an iodinated 1-benzoxepin-2-one compounds.

In some embodiments, which can be used in conjunction with the above aspects and embodiments, the core region comprises a polyol residue.

In some embodiments, which can be used in conjunction with the above aspects and embodiments, the polyamino compound comprises a plurality of basic amino acid residues.

In some embodiments, which can be used in conjunction with the above aspects and embodiments, the system comprises a first composition that comprises the polyamino compound in a first container and a second composition that comprises the reactive multi-arm polymer in a second container.

In some embodiments, which can be used in conjunction with the above aspects and embodiments, the system further comprises a delivery device.

In other aspects, the present disclosure pertains to radiopaque hydrolysable hydrogels that are produced by covalent crosslinking between a reactive multi-arm polymer in accordance with any of the aspects and embodiments and a polyamino compound in accordance with any of the aspects and embodiments.

In some of these embodiments, the radiopaque hydrolysable hydrogel has a radiopacity that is greater than 100 Hounsfield units (HU).

In other aspects, the present disclosure pertains to a method of treatment comprising administering to a subject a mixture that comprises a reactive multi-arm polymer in accordance with any of the aspects and embodiments and a polyamino compound in accordance with any of the aspects and embodiments, wherein the mixture is administered under conditions such that the polyamino compound and the reactive multi-arm polymer react to form a crosslinked hydrogel after administration.

In some embodiments, the method comprises administering to the subject (a) a first fluid composition that comprises the polyamino compound and the reactive multi-arm polymer, and (b) a second fluid composition that comprises an accelerant that accelerates reaction of the polyamino compound and the reactive multi-arm polymer.

Potential benefits associated with the present disclosure include one or more of the following: radiocontrast is maintained or enhanced, crosslink density is tunable, and in vivo persistence is obtained.

The above and other aspects, embodiments, features and benefits of the present disclosure will be readily apparent from the following detailed description.

### BRIEF DESCRIPTION OF THE DRAWINGS

FIG. 1 schematically illustrates the formation of a hydroxyl-terminated 8-arm-polyoxazoline having a tripentaerythritol residue core, in accordance with an embodiment of the present disclosure.

FIG. 2 schematically illustrates the formation of a hydroxyl-terminated 8-arm-polyethylene oxide having a tripentaerythritol residue core, in accordance with an embodiment of the present disclosure.

FIG. 3 schematically illustrates the formation of a hydroxyl-terminated 3-arm-polyethylene oxide having a 1,3,5-triiodo-2,4,6-tris-hydroxymethylbenzene residue core, in accordance with an embodiment of the present disclosure.

FIG. 4 schematically illustrates the formation of an amino-terminated multi-arm-polyethylene oxide, in accordance with an embodiment of the present disclosure.

FIG. 5A schematically illustrates the formation of a multi-arm polyethylene oxide having end groups that comprise hydroxyl-group-containing iodinated lactone residues, in accordance with an embodiment of the present disclosure.

FIG. 5B schematically illustrates the formation of a multi-arm polymer having end groups that comprise hydroxyl-group-containing iodinated lactone residues, in accordance with an embodiment of the present disclosure.

FIG. 6A schematically illustrates the formation of a carboxyl-terminated multi-arm polyethylene oxide, in accordance with an embodiment of the present disclosure.

FIG. 6B schematically illustrates the formation of a carboxyl-terminated multi-arm polymer, in accordance with an embodiment of the present disclosure.

FIG. 7A schematically illustrates the formation of a N-succinimidyloxycarbonyl-terminated multi-arm polyethylene oxide, in accordance with an embodiment of the present disclosure.

FIG. 7B schematically illustrates the formation of a N-succinimidyloxycarbonyl-terminated multi-arm polymer, in accordance with an embodiment of the present disclosure.

FIG. 8 schematically illustrates the formation of a crosslinked polymer network by reaction of trilysine with the multi-arm polymer of FIG. 7A, in accordance with an embodiment of the present disclosure.

FIG. 9 schematically illustrates the hydrolysis of the crosslinked polymer network of FIG. 8, in accordance with an embodiment of the present disclosure.

FIG. 10 schematically illustrates a delivery device, in accordance with an embodiment of the present disclosure.

FIG. 11 schematically illustrates a delivery device, in accordance with another embodiment of the present disclosure.

### DETAILED DESCRIPTION

In some aspects of the present disclosure, radiopaque hydrolysable hydrogels are provided that comprise a crosslinked reaction product of (a) a polyamino compound and (b) a radiopaque reactive polymer comprising reactive moieties that are reactive with the amino groups of the polyamino compound.

As used herein, a “hydrogel” is a crosslinked polymer that contains water or can absorb water but does not dissolve when placed in water.

Radiopaque reactive polymers for use in the present disclosure include reactive multi-arm polymers that comprise a plurality of polymer arms that are linked to a core region. In various embodiments the polymer arms comprise a plurality of arms that each comprise an electrophilic group, a hydrolysable ester group, an iodinated moiety, an amide group and a hydrophilic polymer segment, where the electrophilic group is linked to the iodinated moiety through the hydrolysable ester group, the iodinated moiety is linked to a first end of the hydrophilic polymer segment through the amide group, and a second end (opposite end) of the hydrophilic polymer segment is linked to the core region.

Radiopaque reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms.

Hydrophilic polymer segments for the polymer arms can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, 8-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Polymer segments for use in the multi-arm polymers of the present disclosure typically contain between 10 and 1000 monomer units or more.

Electrophilic groups may be selected, for example, from cyclic imide ester groups (also referred to herein as cyclic-imidyl-oxycarbonyl groups), such as succinimide ester groups,

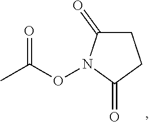

maleimide ester groups, glutarimide ester groups, diglycolimide ester groups, phthalimide ester groups, bicyclo[2.2.1]hept-5-ene-2,3-dicarboxylic acid imide ester groups,

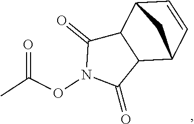 [SMILES: CC(=O)ON1C(=O)C2C(C1=O)[C@H]1C=C[C@@H]2C1]

and tetrahydro-1H-azepine-2,7-dione ester groups,

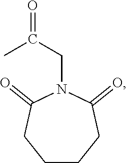

among other possibilities.

Core regions for use in the present disclosure include core regions that comprises a residue of a non-iodinated polyol or an iodinated polyol comprising three or more hydroxyl groups, which is used to form the polymer arms. In certain beneficial embodiments, the core region comprises a residue of a polyol that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxy groups.

In some embodiments of the present disclosure, a non-iodinated polyol or an iodinated polyol such as one of those described below, among others, may be used to form a multi-functional initiator for polymer chain growth.

For example, the non-iodinated or iodinated polyol may be reacted with methanesulfonyl chloride to convert the hydroxyl groups into methanesulfonate groups, which can serve as leaving groups for the cationic polymerization of an oxazoline monomer to form polyoxazoline segments at the sites of each of the hydroxyl groups of the polyol. The resulting hydroxyl-terminated polyoxazoline segments possess tunable hydrophilicity depending on the desired water-solubility of the resulting multi-arm polymer, for example, with increasing polyoxazoline segment length leading to increased hydrophilicity in some embodiments.

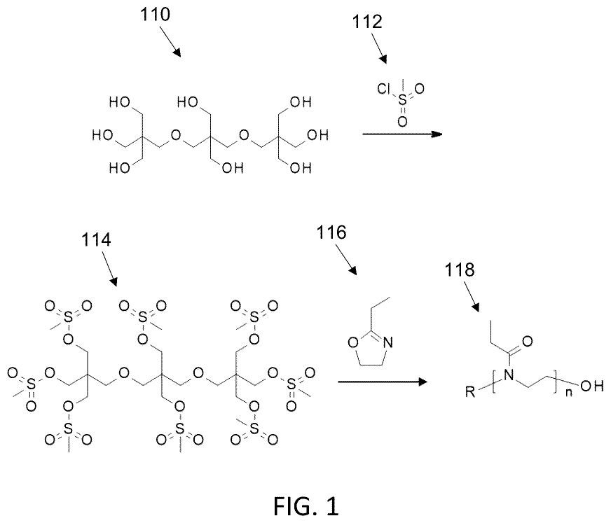

In a particular embodiment shown in FIG. 1, commercially available tripentaerythritol (110) is reacted with methanesulfonyl chloride (112) to convert the hydroxyl groups of the tripentaerythritol into methanesulfonate groups, thereby forming an octa-functional initiator (114), which can be used for the polymerization of 2-ethyl-2-oxazoline (116). The polymerization process leads to poly(2-ethyl-2-oxazoline) chain growth at sites previously occupied by each of the eight hydroxyl groups of the tripentaerythritol, forming a hydroxyl-terminated 8-arm-poly(2-ethyl-2-oxazoline) (118) having a tripentaerythritol residue core and eight poly(2-ethyl-2-oxazoline) arms. Each of the polyoxazoline arms has a terminal hydroxyl group. In FIG. 1, n is an integer representing the number of monomer units in the polymer segment shown. Only a single polymer arm of the hydroxyl-terminated 8-arm-poly(2-ethyl-2-oxazoline) (118) is shown, with R representing the core and the remaining seven arms of the hydroxyl-terminated 8-arm-poly(2-ethyl-2-oxazoline) (118).

As another example, a non-iodinated or iodinated polyol may be used as an initiator for ring-opening polymerization of ethylene oxide to form polyethylene oxide (PEO) segments (also referred to a polyethylene glycol (PEG) segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity.

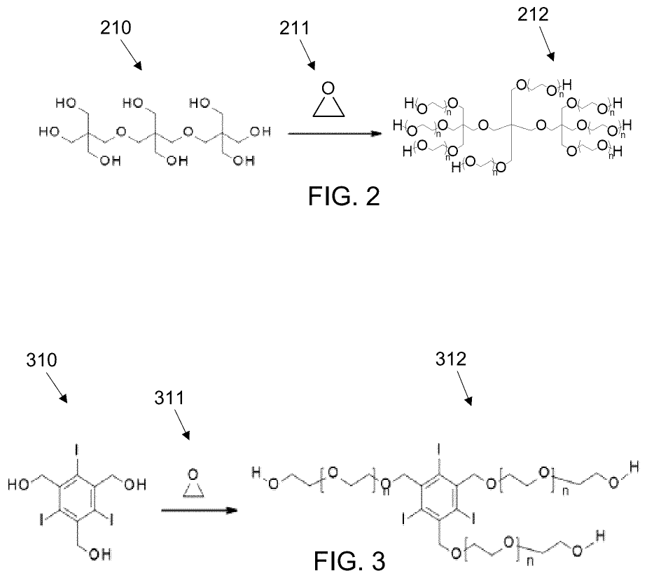

In a particular embodiment shown in FIG. 2, commercially available tripentaerythritol (210) is used as an octa-functional initiator, which undergoes ring-opening polymerization with ethylene oxide (211). The polymerization process leads to poly(ethylene oxide) chain growth at each of the eight hydroxyl groups of the tripentaerythritol, forming an 8-arm-PEG (212) having a tripentaerythritol residue core. Each of the PEG arms has a terminal hydroxyl group. In FIG. 2, n is an integer representing the number of monomer units in each polymer segment shown.

The strategies shown in FIGS. 1 and 2 can be used in conjunction with a wide range of polyols, including those described below. In another particular embodiment shown in FIG. 3, an iodinated polyol, specifically, 1,3,5-triiodo-2,4,6-tris-hydroxymethylbenzene (310), is used as an initiator, which undergoes ring-opening polymerization with ethylene oxide (311). The polymerization process leads to poly(ethylene oxide) (PEG) chain growth at each of the three hydroxyl groups of the 1,3,5-triiodo-2,4,6-tris-hydroxymethylbenzene (310). The resulting multi-arm polymer (312) contains three PEG arms that extend from a 1,3,5-triiodo-2,4,6-tris-hydroxymethylbenzene residue core. Each of the PEG arms has a terminal hydroxyl group. In FIG. 3, n is an integer representing the number of monomer units in each polymer segment shown.

Illustrative non-iodinated polyols may be selected, for example, from sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Illustrative non-iodinated polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Illustrative non-iodinated polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

Iodinated polyols are desirable where additional radiopacity is desired. Illustrative iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, ether groups, alkyl groups, and combinations thereof, among others.

Further illustrative iodinated polyols for use in the present disclosure, in addition to the 1,3,5-triiodo-2,4,6-tris-hydroxymethylbenzene described above, include iodinated polyols that are known for use as iodinated contrast agents, whose biocompatibility has been demonstrated to be reasonably well tolerated. Specific examples of iodinated polyols include commercially available 1,3,5-triiodo-2,4,6-trishydroxymethylbenzene (CAS #178814-33-0), iodixanol (CAS #92339-11-2), iotrolan (CAS #79770-24-4), iohexol (CAS #66108-95-0), ioversol (CAS #87771-40-2), iopamidol (CAS #60166-93-0), iohexol impurity J (CAS #76801-93-9), and iopromide (CAS #73334-07-3), among others.

Reactive multi-arm polymers in accordance with the present disclosure can be formed from hydroxyl-terminated multi-arm polymers through various synthetic routes.

In some embodiments, in an initial step, hydroxyl end groups of a hydroxyl-terminated multi-arm polymer converted into amino groups of an amino-terminated multi-arm polymer having arms that each comprise one or more amino end groups.

For example, a multi-arm polymer that comprises a core region and a plurality of polymer arms with hydroxyalkyl end groups may be reacted with methanesulfonyl chloride to form a multi-arm polymer that comprises a core region and a plurality of polymer arms with methanesulfonate end groups. The methanesulfonate end groups may then be reacted with ammonia to form a multi-arm polymer that comprises a core region and a plurality of polymer arms with amino end groups.

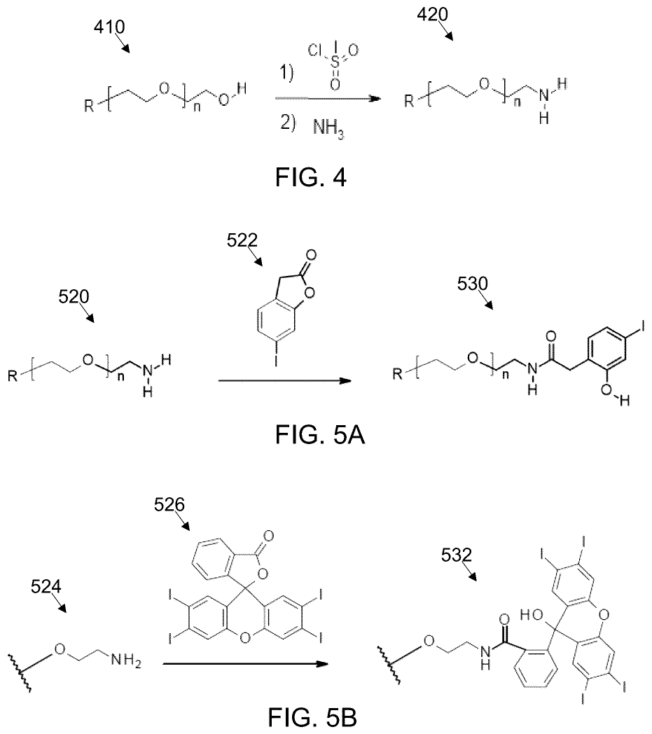

In a particular example shown in FIG. 4. a hydroxyl-terminated multi-arm PEG (410) having a polyol core, where n represents the number monomer units (note that only one polymer arm is shown; the polyol residue and the remaining arms are represented by R), for example, a hydroxyl-terminated 8-arm PEG (410) like that of FIG. 2, where R may comprise, for example, a tripentaerythritol or hexaglycerol residue and n ranges from 30 to 140, is first treated with methanesulfonyl chloride to form a polymer in which hydroxyl groups of the 8-arm PEG (410) are converted into methanesulfonate groups. The methanesulfonate groups are then reacted with ammonia to convert the methanesulfonate groups to amino groups, thereby forming an amino-terminated multi-arm (520), for example an amino-terminated 8-arm PEG. Analogous processes can be performed with other hydroxyl-terminated multi-arm polymers, including those of FIG. 1 and FIG. 3, among many others.

Various amino-terminated multi-arm polymers are also available commercially. For example, amino-terminated four-arm PEG,

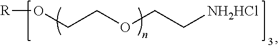

amino-terminated four-arm PEG,

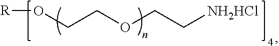

where R represents a pentaerythritol residue core, amino-terminated six-arm PEG,

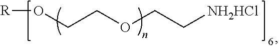

where R represents a dipentaerythritol residue core, and amino-terminated eight-arm PEG,

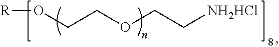

where R represents a tripentaerythritol residue core, are available from JenKem Technology USA, Plano, TX, USA. The preceding amino-terminated multi-arm polymers are shown in salt form, specifically, in the hydrochloride salt form.

After obtaining an amino-terminated multi-arm polymer, the amino-terminated multi-arm polymer may then be reacted with an iodinated lactone compound (also referred to as an iodinated cyclic carboxylic acid ester) in a ring-opening reaction to form a hydroxy-terminated multi-arm polymer in which each arm comprises an iodinated moiety that is linked to a hydrophilic polymer segment through an amide group, more particularly, a hydroxy-terminated multi-arm polymer in which each arm comprises an iodinated lactone residue that is linked to a hydrophilic polymer segment through an amide group.

Examples of iodinated lactone compounds include iodinated benzolactone compounds such as iodinated 1-benzofuran-2-one compounds, iodinated 2-benzofuran-1-one compounds, iodinated chroman-2-one compounds, iodinated 1-benzoxepin-2-one compounds, iodinated 1-benzopyran-2-one compounds, iodinated 2-benzopyran-3-one compounds, iodinated 2-benzopyran-1-one compounds, and among others.

Particular examples of iodinated 1-benzofuran-2-one compounds include mono-iodo-3H-1-benzofuran-2-one compounds such as 4-iodo-3H-1-benzofuran-2-one, 5-iodo-3H-1-benzofuran-2-one,

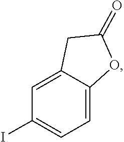 [SMILES: O=C1Cc2cc(I)ccc2O1]

6-iodo-3H-1-benzofuran-2-one,

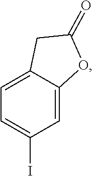 [SMILES: O=C1Cc2ccc(I)cc2O1]

and 7-iodo-3H-1-benzofuran-2-one, diiodo-3H-1-benzofuran-2-one compounds such as 5,6-diiodo-3H-1-benzofuran-2-one, 5,7-diiodo-3H-1-benzofuran-2-one,

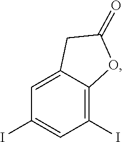 [SMILES: O=C1Cc2cc(I)cc(I)c2O1]

4,5-diiodo-3H-1-benzofuran-2-one, 6,7-diiodo-3H-1-benzofuran-2-one, 4,6-diiodo-3H-1-benzofuran-2-one and 4,7-diiodo-3H-1-benzofuran-2-one, triiodo-3H-1-benzofuran-2-one compounds such as 5,6,7-triiodo-3H-1-benzofuran-2-one, 4,5,6-triiodo-3H-1-benzofuran-2-one, 4,5,7-triiodo-3H-1-benzofuran-2-one, and 4,6,7-tri-iodo-3H-1-benzofuran-2-one, and tetraiodo-3H-1-benzofuran-2-one compounds such as 4,5,6,7-tetraiodo-3H-1-benzofuran-2-one, among others.

Particular examples of iodinated 2-benzofuran-1-one compounds include mono-iodo-3H-2-benzofuran-1-one compounds such as 4-iodo-3H-2-benzofuran-1-one, 5-iodo-3H-2-benzofuran-1-one, 6-iodo-3H-2-benzofuran-1-one,

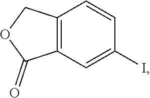 [SMILES: O=C1OCc2ccc(I)cc21]

and 7-iodo-3H-2-benzofuran-1-one, diiodo-3H-2-benzofuran-1-one compounds such as 5,6-diiodo-3H-2-benzofuran-1-one, 5,7-diiodo-3H-2-benzofuran-1-one, 4,5-diiodo-3H-2-benzofuran-1-one, 6,7-diiodo-3H-2-benzofuran-1-one, 4,6-diiodo-3H-2-benzofuran-1-one and 4,7-diiodo-3H-2-benzofuran-1-one, triiodo-3H-2-benzofuran-1-one compounds such as 5,6,7-triiodo-3H-2-benzofuran-1-one, 4,5,6-triiodo-3H-2-benzofuran-1-one, 4,5,7-triiodo-3H-2-benzofuran-1-one, and 4,6,7-triiodo-3H-2-benzofuran-1-one, and tetraiodo-3H-2-benzofuran-1-one compounds such as 4,5,6,7-tetraiodo-3H-2-benzofuran-1-one, among others. Iodinated 2-benzofuran-1-one compounds further include, 2′,3′,6′, 7′-tetraiodo-Spiro[isobenzofuran-1 (3H),9′-[9H]xanthen]-3-one,

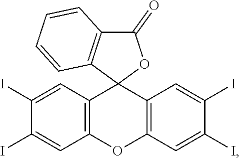 [SMILES: O=C1OC2(c3cc(I)c(I)cc3Oc3cc(I)c(I)cc32)c2ccccc21]

and, 2′,4′,5′,7′-tetraiodo-spiro[isobenzofuran-1 (3H),9′-[9H]xanthen]-3-one,

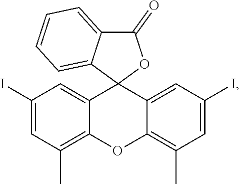 [SMILES: Cc1cc(I)cc2c1Oc1c(C)cc(I)cc1C21OC(=O)c2ccccc21]

among others.

Particular examples of iodinated chroman-2-one compounds include mono-iodo-chroman-2-ones, such as 5-iodo-chroman-2-one,

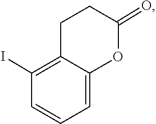 [SMILES: O=C1CCc2c(I)cccc2O1]

6-iodo-chroman-2-one, 7-iodo-chroman-2-one, and 8-iodo-chroman-2-one, diiodo-chroman-2-ones such as 5,6-diiodo-chroman-2-one, 5,7-diiodo-chroman-2-one, 5,8-diiodo-chroman-2-one, 6,7-diiodo-chroman-2-one, 6,8-diiodo-chroman-2-one, and 7,8-diiodo-chroman-2-one, triiodo-chroman-2-ones such as 5,6,7-triiodo-chroman-2-one, 5,6,8-triiodo-chroman-2-one, 5,7,8-triiodo-chroman-2-one, and 6,7,8-triiodo-chroman-2-one, and tetra-iodo-chroman-2-ones such as 5,6,7,8-tetraiodo-chroman-2-one.

Particular examples of iodinated 1-benzoxepin-2-one compounds include mono-iodo-4,5-dihydro-3H-1-benzoxepin-2-ones such as 6-iodo-4,5-dihydro-3H-1-benzoxepin-2-one, 7-iodo-4,5-dihydro-3H-1-benzoxepin-2-one, 8-iodo-4,5-dihydro-3H-1-benzoxepin-2-one, and 9-iodo-4,5-dihydro-3H-1-benzoxepin-2-one,

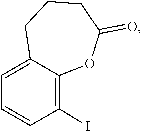 [SMILES: O=C1CCCc2cccc(I)c2O1]

diiodo-4,5-dihydro-3H-1-benzoxepin-2-ones such as 6,7-diiodo-4,5-dihydro-3H-1-benzoxepin-2-one, 6,8-diiodo-4,5-dihydro-3H-1-benzoxepin-2-one, 6,9-diiodo-4,5-dihydro-3H-1-benzoxepin-2-one, 7,8-diiodo-4,5-dihydro-3H-1-benzoxepin-2-one, 7-9-diiodo-4,5-dihydro-3H-1-benzoxepin-2-one, and 8,9-diiodo-4,5-dihydro-3H-1-benzoxepin-2-one, triiodo-4,5-dihydro-3H-1-benzoxepin-2-ones such as 6,7,8-triiodo-4,5-dihydro-3H-1-benzoxepin-2-one, 6,7,9-triiodo-4,5-dihydro-3H-1-benzoxepin-2-one, 6,8,9-triiodo-4,5-dihydro-3H-1-benzoxepin-2-one, and 7,8,9-triiodo-4,5-dihydro-3H-1-benzoxepin-2-one, and tetra-iodo-4,5-dihydro-3H-1-benzoxepin-2-ones such as 6,7,8,9-tetraiodo-4,5-dihydro-3H-1-benzoxepin-2-one.

In a particular example shown in FIG. 5A, an amino-terminated multi-arm polymer wherein the polymer arms have terminal amino groups, specifically, an amino-terminated multi-arm PEG (520) having a polyol core, where n represents the number monomer units (note that only one polymer arm is shown; the polyol residue and the remaining arms are represented by R), for example, an amino-terminated multi-arm PEG like that of FIG. 4, is reacted with an iodinated lactone compound, specifically, 6-iodo-3H-1-benzofuran-2-one (522) in a ring-opening reaction. The product of the ring-opening reaction is a multi-arm polymer comprising a plurality of polymer arms having an end group that comprises a hydroxyl-group-containing iodinated lactone residue that is linked to the hydrophilic polymer segment of the polymer arm through an amide group, more particularly, a multi-arm PEG (530) comprising a plurality of polymer arms having an end group that comprises a hydroxyl-group-containing 6-iodo-3H-1-benzofuran-2-one residue that is linked to the PEG segment of the arm through an amide group.

In another particular example shown in FIG. 5B, an amino-terminated multi-arm polymer wherein the polymer arms have terminal amino groups (525), for example, an amino-terminated multi-arm PEG like that of FIG. 4, an amino-terminated multi-arm polyoxazoline, or another amino-terminated multi-arm polymer (note that only a single end group of a single polymer arm is shown), is reacted with an iodinated lactone compound, specifically, 2′,3′,6′,7′ tetraiodo-spiro[isobenzofuran-1 (3H),9′. [9H]xanthen]-3-one (526) in a ring-opening reaction. The product of the ring-opening reaction is a multi-arm polymer comprising a plurality of polymer arms having an end group that comprises a hydroxyl-group-containing iodinated lactone residue that is linked to the hydrophilic polymer segment of the polymer arm through an amide group, more particularly, a multi-arm polymer (532) comprising a plurality of polymer arms having an end group that comprises an iodinated spiro isobenzofurane residue that is linked to the hydrophilic polymer segment of the arm through an amide group.

The strategy shown in FIGS. 5A and 5B is widely applicable to other amino-terminated polymers and other iodinated lactones beyond those illustrated. In each case, a multi-arm polymer will be formed having polymer arms that comprise a iodinated end group, specifically an end group comprising an iodinated lactone residue that comprises a hydroxy group and one or more iodine groups, which is linked to the hydrophilic polymer segment of the polymer arm through an amide group.

The hydroxyl group can be used to form a hydrolysable ester group. For example, in some embodiments, the hydroxyl group is reacted with a cyclic anhydride in a ring opening reaction to form a carboxylic-acid-terminated multi-arm polymer in which the carboxylic acid groups are linked to the iodinated lactone residues of the polymer arms through linkages that comprises hydrolysable ester groups.

Cyclic anhydrides for use in the present disclosure include, for example, various cyclic anhydrides having a ring that comprises a —C₂-C₈-alkylene-group, including, for example, succinic anhydride,

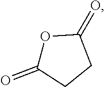

which comprises a dimethylene group, glutaric anhydride,

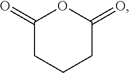

which comprises a trimethylene group, adipic anhydride,

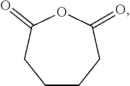

which comprises a tetramethylene group, pimelic anhydride,

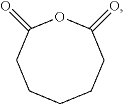

which comprises a pentamethylene group, and suberic anhydride,

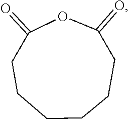 [SMILES: O=C1CCCCCCC(=O)O1]

which comprises a hexamethylene group, among others. Further anhydrides are listed in the following table.

| Species | CAS # |
|---|---|
| Succinic anhydride | 108-30-5 |
| Glutaric anhydride | 108-55-4 |
| Adipic anhydride | 2035-75-8 |
| Pimelic anhydride | 10521-07-0 |
| Malonic anhydride | 15159-48-5 |
| Itaconic anhydride | 2170-03-8 |
| Maleic anhydride | 108-31-6 |
| 3-Oxabicyclo[3.1.0]hexane-2,4-dione | 5617-74-3 |
| 3-Oxabicyclo[3.2.0]heptane-2,4-dione | 4462-96-8 |
| 3-Methylglutaric anhydride | 4166-53-4 |
| Methylsuccinic anhydride | 4100-80-5 |
| 2,2-Dimethylsuccinic anhydride | 17347-61-4 |
| 6,6-Dimethyl-3-oxabicyclo[3.1.0]hexane-2,4-dione | 67911-21-1 |
| Dihydro-4,4-dimethyl-2H-pyran-2,6(3H)-dione | 4160-82-1 |
| Diglycolic anhydride | 4480-83-5 |
| Oxane-2,4,6-trione | 10521-08-1 |
| Dihydro-4-(2-methylpropyl)-2H-pyran-2,6(3H)-dione | 185815-59-2 |

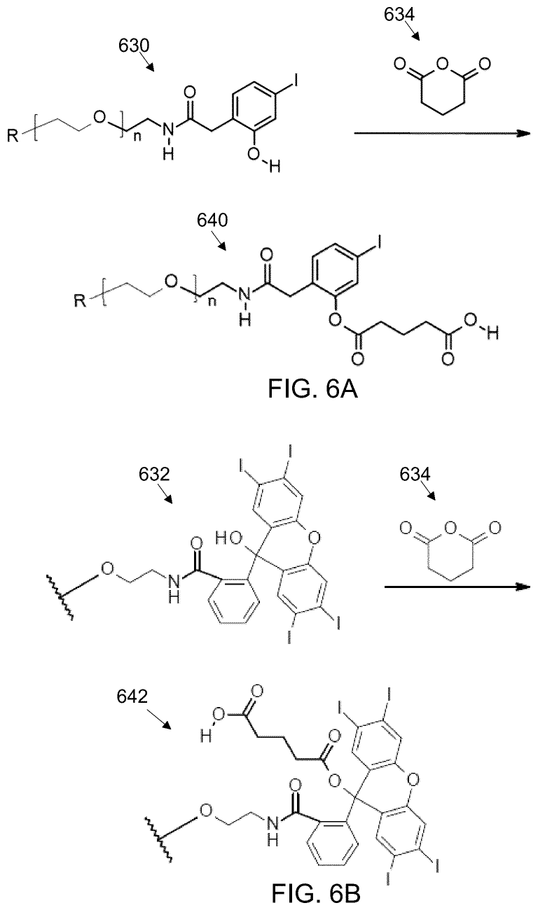

Turning now to FIG. 6A, a multi-arm polymer having polymer arms with end groups that comprise an iodinated lactone residue that is linked to the hydrophilic polymer segment of the arm through an amide group, specifically, a multi-arm PEG (630) comprising a plurality of polymer arms having an end group that comprises a 6-iodo-3H-1-benzofuran-2-one residue that is linked to the PEG segment of the arm through an amide group, is reacted in a ring-opening reaction with a cyclic anhydride, specifically, glutaric anhydride (634), to form a carboxylic-acid-terminated multi-arm polymer having a plurality of arms in which a carboxylic acid group is linked to an iodinated lactone residue of the polymer arm through a linkage that comprises a hydrolysable ester group, more particularly, a carboxylic-acid-terminated multi-arm PEG (640) having a plurality of arms in which a carboxylic acid group is linked to a 6-iodo-3H-1-benzofuran-2-one residue through a trimethylene ester group.

Similarly, in FIG. 6B, a multi-arm polymer (632) comprising a plurality of polymer arms having an end group that comprises an iodinated spiro[isobenzofuran xanthen]one residue that is linked to the hydrophilic polymer segment of the arm through an amide group, is reacted in a ring-opening reaction with glutaric anhydride (634) to form a carboxylic-acid-terminated multi-arm polymer (642) having a plurality of arms in which a carboxylic acid group is linked to an iodinated xanthene residue through a trimethylene ester group.

As can be appreciated from the above, when a cyclic anhydride having a ring that comprises a —C₂-C₈-alkylene-group is reacted with a iodinated-lactone-terminated multi-arm polymer, the result is a multi-arm polymer having carboxyl-C₂-C₈-alkylenecarbonyloxy-groups, for example, a carboxydimethylenecarbonyloxy-group in the case of succinic anhydride, a carboxytrimethylenecarbonyloxy-group in the case of glutaric anhydride, a carboxytetramethylenecarbonyloxy-group in the case of adipic anhydride, a carboxypentamethylenecarbonyloxy-group in the case of pimelic anhydride, a carboxyhexamethylenecarbonyloxy-group in the case of suberic anhydride, and so forth.

After forming a carboxylic-acid-terminated multi-arm polymer, an electrophilic group may be formed at the site of each of the carboxylic acid groups of the carboxylic-acid-terminated multi-arm polymer.

For example, in particular embodiments where a cyclic imide ester group is employed as an electrophilic group, an N-hydroxy cyclic imide compound may be reacted with the carboxylic acid groups of the carboxylic-acid-terminated multi-arm polymer in an amide coupling reaction to form a reactive multi-arm polymer in which reactive cyclic imide ester groups are linked to iodinated lactone residues in the polymer arms through a linkage that comprises a hydrolysable ester group.

For instance, an N-hydroxy cyclic imide compound (e.g., N-hydroxysuccinimide, N-hydroxymaleimide, N-hydroxyglutarimide, N-hydroxyphthalimide, N-hydroxydiglycolimide or N-hydroxy-5-norbornene-2,3-dicarboxylic acid imide, also known as N-hydroxybicyclo[2.2.1]hept-5-ene-2,3-dicarboxylic acid imide (HONB), tetrahydro-1-hydroxy-1H-azepine-2,7-dione,

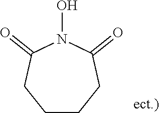

may be reacted with the carboxylic acid groups of the carboxylic-acid-terminated polymer in the presence of a suitable coupling agent (e.g., a carbodiimide coupling agent, such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester group (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to an iodinated lactone residue through a linkage that comprises an ester group. In this way, a variety of reactive ester groups can be formed.

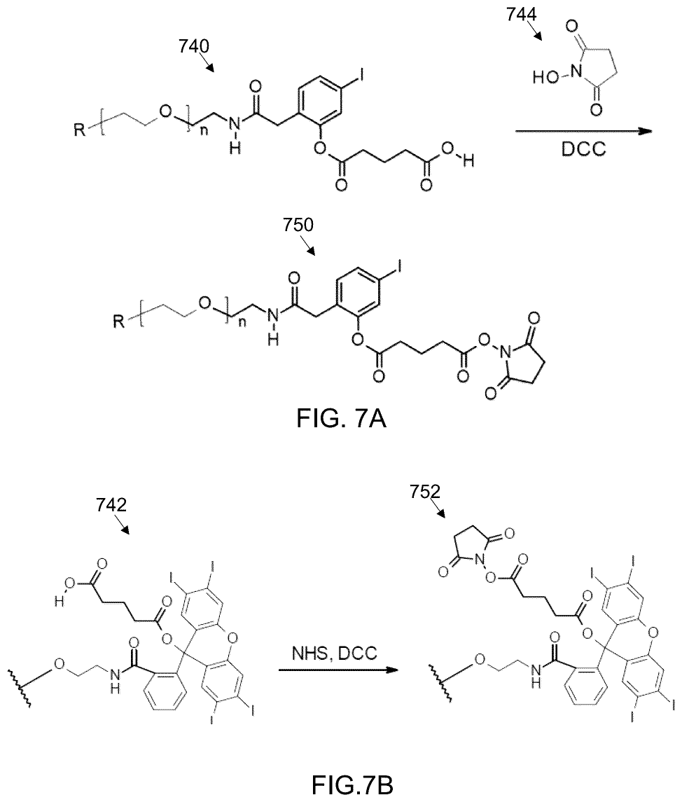

In a particular example shown in FIG. 7A, carboxylic acid groups of a carboxylic-acid-terminated multi-arm polymer, specifically, a carboxylic-acid-terminated multi-arm PEG (740) having a plurality of arms in which carboxylic acid groups are linked to 6-iodo-3H-1-benzofuran-2-one residues through trimethylene ester groups are reacted with an N-hydroxy cyclic imide compound, specifically N-hydroxy succinimide (744) in an amide coupling reaction in the presence of a suitable coupling agent such as DCC to form cyclic-imidyl-ester-terminated multi-arm polymer, specifically a succinimide-ester-terminated multi-arm PEG (750) in which the succinimide ester groups are linked to the 6-iodo-3H-1-benzofuran-2-one residues of the multi-arm polymer through linkages that comprise a three carbon alkyl chain (i.e., a trimethylene group) and an ester group.

Similarly, as shown in FIG. 7B, carboxylic acid groups of a carboxylic-acid-terminated multi-arm polymer, specifically, a carboxylic-acid-terminated multi-arm polymer (742) having a plurality of arms in which a carboxylic acid group is linked to a iodinated xanthene residue through a trimethylene ester group, are reacted with an N-hydroxy cyclic imide compound, specifically N-hydroxy succinimide (NHS) in an amide coupling reaction in the presence of a suitable coupling agent such as DCC to form succinimide-ester-terminated multi-arm polymer (752) in which the succinimide ester groups are linked to the iodinated xanthene residues of the multi-arm polymer through linkages that comprise a three carbon alkyl chain (i.e., a trimethylene group) and an ester group.

As can be seen from FIGS. 7A and 7B, where the carboxylic-acid-terminated multi-arm polymer is formed using glutaric anhydride, the resulting cyclic-imidyl-ester-terminated multi-arm polymer comprises cyclic-imidyl-oxycarbonyl-1,3-n-propylene-carbonyloxy-end groups, more specifically succinimidyl-oxycarbonyl-1,3-n-propylene-carbonyloxy-end groups. As can be appreciated from this example, various cyclic anhydrides having a ring that comprises a —C₂-C₈-alkylene-group may be used to form various cyclic-imidyl-oxycarbonyl-C₂-C₆-alkylene-carbonyloxy-end groups. For example, (a) succinic anhydride can be used to form cyclic-imidyl-oxycarbonyl-1,2-ethylene-carbonyloxy-end groups, more specifically succinimidyl-oxycarbonyl-1,2-ethyl-carbonyloxy-end groups, (b) adipic anhydride can be used to form cyclic-imidyl-oxycarbonyl-1,4-n-butylene-carbonyloxy-end groups, more specifically succinimidyl-oxycarbonyl-1,4-n-butylene-carbonyloxy-end groups, (c) pimelic anhydride can be used to form cyclic-imidyl-oxycarbonyl-1,5-n-pentylene-carbonyloxy-end groups, more specifically succinimidyl-oxycarbonyl-1,5-n-pentylene-carbonyloxy-end groups, and (d) suberic anhydride can be used to form cyclic-imidyl-oxycarbonyl-1,6-n-hexylene-carbonyloxy-end groups, more specifically succinimidyl-oxycarbonyl-1,6-n-hexylene-carbonyloxy-end groups, among other possibilities.

Examples of cyclic-imidyl-oxycarbonyl-C₂-C₈-alkylene-carbonyloxy groups include succinimidyl-oxycarbonyl-C₂-C₈-alkylene-carbonyloxy-groups, maleimidyl-oxycarbonyl-C₂-C₈-alkylene-carbonyloxy-groups, glutarimidyl-oxycarbonyl-C₂-C₈-alkylene-carbonyloxy-groups, diglycolimidyl-oxycarbonyl-C₂-C₈-alkylene-carbonyloxy-groups, phthalimidyl-oxycarbonyl-C₂-C₈-alkylene-carbonyloxy-groups, and bicyclo[2.2.1]hept-5-ene-2,3-dicarboxylic-acid-imidyl-oxycarbonyl-C₂-C₈-alkylene-carbonyloxy groups-, among other possibilities.

As previously noted, in some aspects, the present disclosure provides radiopaque hydrolysable crosslinked hydrogels that comprise a radiopaque hydrolysable crosslinked reaction product of (a) a polyamino compound and (b) a radiopaque reactive polymer, which may be selected from those described above, that comprises electrophilic groups, which are reactive with amino groups of the polyamino compound.

Polyamino compounds suitable for use in the present disclosure include, for example, polyamines that contain at least two amino (—NH₂) groups (e.g., 2, 3, 4, 5, 6, 7, 8, 9, 10 or more amino groups in some embodiments). Polyamino compounds suitable for use in the present disclosure include those that comprise a plurality of —(CH₂)ₓ—NH₂ groups where x is 0, 1, 2, 3, 4, 5 or 6. Polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further particular examples of polyamino compounds which may be used as the multifunctional compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Where additional radiopacity is desired in the crosslinked hydrogel, iodinated polyamine compounds may be employed.

As previously noted, in some aspects, the present disclosure provides hydrogels that comprise a radiopaque hydrolysable crosslinked reaction product of (a) a polyamino compound, such as those described above, and (b) a radiopaque reactive polymer comprising reactive moieties that are reactive with amino groups of the polyamino compound, such as those described above.

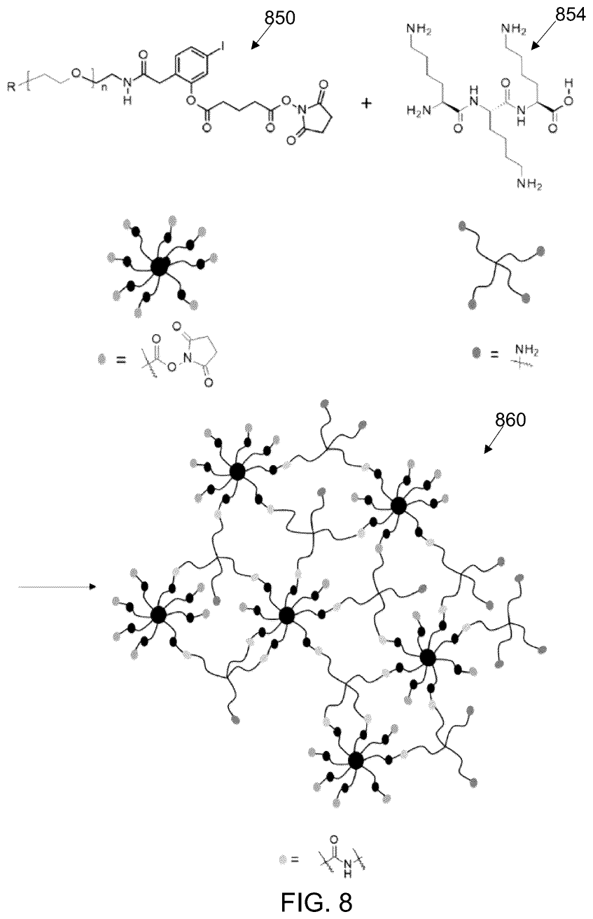

A specific embodiment is shown in FIG. 8, which schematically illustrates a crosslinking reaction between a radiopaque reactive polymer (850) like that of FIG. 7A and a polyamino compound, specifically, trilysine (854). The radiopaque reactive polymer (850) and the polyamino compound (854) are combined under conditions such that the succinimidyl ester groups of the radiopaque reactive polymer (850) react with the amino groups of the polyamino compound (854) to form amide bonds accompanied by the release of N-hydroxysuccinimide, with the result being a crosslinked polymer network (860). In certain embodiments, reaction between the succinimidyl ester groups and the amino groups is conducted at slightly basic pH (e.g., having a pH value ranging from 7.4 to 11) where the amino groups are deprotonated/neutrally charged and amide bond formation can occur spontaneously at body temperature.

It will be appreciated that the crosslinking density of the radiopaque hydrolysable crosslinked hydrogels described herein can be tuned, for example, by varying the number of amino groups in the polyamino compound, by varying the number of arms of the reactive multi-arm polymer, or both.

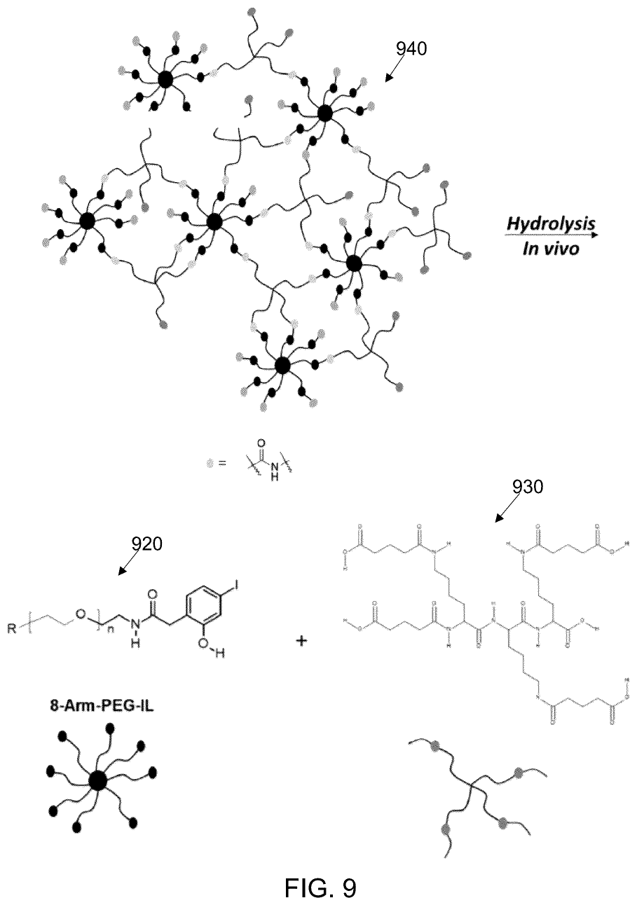

The crosslinked polymer network (860) contains ester groups that can be hydrolyzed in vivo. For example, because reactive polymer (850) that is used to form the crosslinked polymer network (860) contains a cyclic-imidyl-oxycarbonyl-C₂-C₈-alkylene-carbonyloxy-groups, more particularly, cyclic-imidyl-oxycarbonyl-trimethylene-carbonyloxy-groups, the resulting reaction product contains-aminocarbonyl-C₂-C₈-alkylene-carbonyloxy-linkages, more particularly,-aminocarbonyl-trimethylene-carbonyloxy-linkages, through which iodinated lactone residues of the radiopaque reactive polymer are linked to residues of the polyamine. As seen from FIG. 9, upon hydrolysis of the carbonyloxy (ester) linkages, a crosslinked polymer network (940) like that of FIG. 8 will break down into (a) a multi-arm polymer (920) like that shown in FIG. 5A, which contains hydroxyl-group-containing iodinated lactone residues that are linked to the hydrophilic polymer segment of the polymer arm through an amide group and (b) a derivative of the polyamino compound in which amino groups are replaced with carboxy-C₂-C₈-alkylene-carbonylamino-groups, specifically, a trilysine derivative in which side chains of the trilysine residue are terminated with carboxy-C₂-C₈-alkylene-carbonylamino-groups, more specifically, carboxy-trimethylene-carbonylamino-groups.

It is noted that the breakdown products of the present disclosure contain a multi-arm polymer in which iodinated lactone residues are linked to hydrophilic polymer segments through an amide group, which does not hydrolyze. As a result, the lactone residues are securely linked to the polymer segments of the multi-arm polymer breakdown product, rather than being linked to a residue of the polyamino compound. Because iodinated lactone residues are linked to a multi-arm polymer, the radiopacity of the breakdown product persists longer than would occur if the iodinated lactone resides were released or were attached a lower molecular weight polyamino compound residue. There will not be any small molecules of iodinated species generated after hydrolysis, and vice versa.

In some embodiments, the radiopaque hydrolysable crosslinked hydrogels of the present disclosure are visible under fluoroscopy. The radiopaque hydrolysable crosslinked hydrogels may have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values), for example, when measured on bench-top micro CT systems such as Xtreme CT from Scanco Medical (Wangen-Brüttisellen, Switzerland) or similar.

In some aspects of the present disclosure, a system is provided that comprises (a) a first composition that comprises a polyamino compound, as described herein, and (b) a second composition that comprises a radiopaque reactive polymer, wherein the system is configured to deliver the radiopaque reactive polymer and the polyamino compound under conditions such that covalent crosslinks are formed between the radiopaque reactive polymer and the polyamino compound.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below. The second composition may be a second fluid composition comprising the radiopaque reactive polymer or a second dry composition that comprises the radiopaque reactive polymer, to which a suitable fluid such as water for injection, saline, etc. can be added to form a second fluid composition. In addition to the radiopaque reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the polyamino compound with a second fluid comprising the radiopaque reactive polymer. Upon mixing the first and second fluid compositions, the polyamino compound crosslinks with the radiopaque reactive polymer, forming a crosslinked product. The first and second fluid compositions may be combined to form radiopaque hydrolysable crosslinked hydrogels.

In some embodiments, the polyamino compound is initially combined with the radiopaque reactive polymer under conditions where crosslinking between the radiopaque reactive polymer and the polyamino compound is suppressed (e.g., an acidic pH in the case where the radiopaque reactive polymer comprises reactive electrophilic moieties). Then, when crosslinking is desired, the conditions are changed such that crosslinking is increased (e.g., a change from an acidic pH to a basic pH, in some embodiments), leading to crosslinking between the polyamino compound and the radiopaque reactive polymer, thereby forming a crosslinked product. The first and second fluid compositions may be combined to form radiopaque hydrolysable crosslinked hydrogels.

In some of these embodiments, the system comprises (a) a first composition that comprises a polyamino compound as described hereinabove, (b) a second composition that comprises a radiopaque reactive polymer as described hereinabove, and (c) a third composition, specifically, an accelerant composition, that contains an accelerant that is configured to accelerate a crosslinking reaction between the polyamino compound and the radiopaque reactive polymer.

The first composition may be a first fluid composition comprising the polyamino compound that is buffered to an acidic pH or a first dry composition that comprises the polyamino compound, to which a suitable fluid such as water for injection, saline, an acidic buffer solution, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 6.5, typically, from about 3 to about 5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the radiopaque reactive polymer or a second dry composition that comprises the radiopaque reactive polymer from which a fluid composition is formed, for example, by the addition of a suitable fluid such as water for injection, saline, or the first fluid composition comprising the polyamino compound that is buffered to an acidic pH. In addition to the radiopaque reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the radiopaque reactive polymer. The first composition may then be mixed with the second composition to provide a prepared fluid composition that is buffered to an acidic pH and comprises the polyamino compound and the radiopaque reactive polymer. In a particular example, a syringe may be provided that contains the first fluid composition comprising the polyamino compound that is buffered to an acidic pH, and a vial may be provided that comprises the dry composition (e.g., a powder) that comprises the radiopaque reactive polymer. The syringe may then be used to inject the first fluid composition into the vial containing the radiopaque reactive polymer to form a prepared fluid composition that is buffered to an acidic pH and contains the polyamino compound and the radiopaque reactive polymer, which can be withdrawn back into the syringe for administration.

The accelerant composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 12, typically, from about 9 to about 11. In addition to the above, the fluid accelerant composition may further comprise additional agents, including those described below.

A prepared fluid composition that is buffered to an acidic pH and comprises polyamino compound and radiopaque reactive polymer as described above, and a fluid accelerant composition that is buffered to basic pH as described above, may be combined to form radiopaque hydrolysable crosslinked hydrogels.

As previously noted, additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotic agents, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111 In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a polyamino compound as described herein and a second reservoir that contains a second fluid composition that comprises a radiopaque reactive polymer as described herein, wherein the first and second fluid compositions form a crosslinked product upon mixing.

In some embodiments, the system may include a delivery device that comprises a first reservoir that contains a first fluid composition that comprises a polyamino compound as described herein and a radiopaque reactive polymer as described herein and is buffered to an acidic pH, such as the prepared fluid composition previously described, and a second reservoir that contains second fluid composition, such as the fluid accelerant composition described herein.

In either case, during operation, the first fluid composition and the second fluid composition are dispensed from the first and second reservoirs and combined, whereupon the polyamino compound and the radiopaque reactive polymer and crosslink with one another to form a radiopaque hydrolysable crosslinked hydrogel.

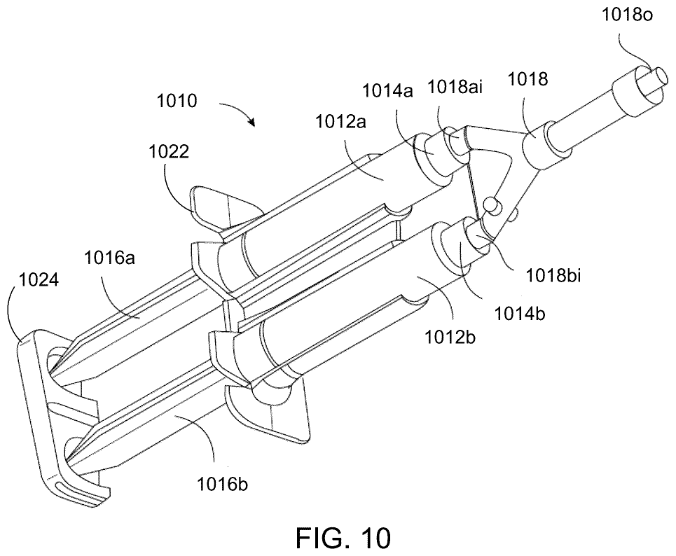

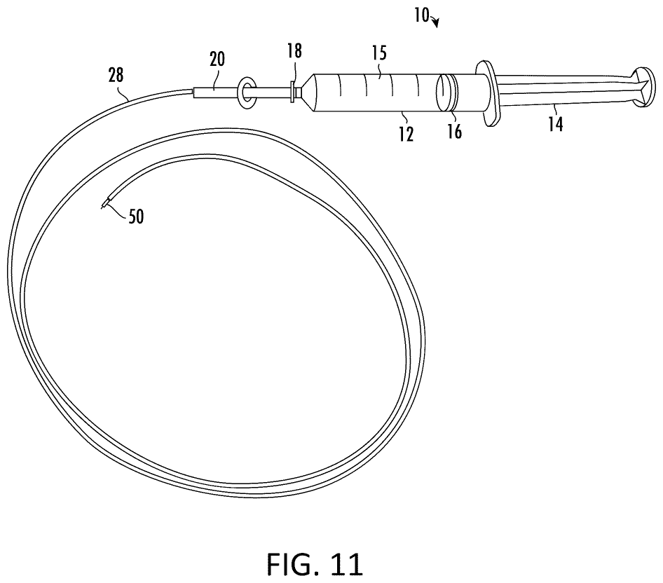

In particular embodiments, and with reference to FIG. 10, the system may include a delivery device 1010 that comprises a double-barrel syringe, which includes a first barrel 1012a having a first barrel outlet 1014a, which first barrel contains a first fluid composition as described above, a first plunger 1019a that is movable in the first barrel 1012a, a second barrel 1012b having a second barrel outlet 1014b, which second barrel 1012b contains a second fluid composition as described above, and a second plunger 1019b that is movable in the second barrel 1012b. In some embodiments, the device 1010 may further comprise a mixing section 1018 having a first mixing section inlet 1018ai in fluid communication with the first barrel outlet 1014a, a second mixing section inlet 1018bi in fluid communication with the second barrel outlet 1014b, and a mixing section outlet 10180. Also shown are a syringe holder 1022 configured to hold the first and second syringe barrels 1012a, 1012b, in a fixed relationship and a plunger cap 1024 configured to hold the first and second plungers 1019a, 1019b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a radiopaque hydrolysable crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of system that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, in some embodiments, the first and second fluid compositions or a fluid admixture thereof can be injected as a bulking agent, for instance, into tissue around the ureteral orifices for the treatment of vesicoureteral reflux, the first and second fluid compositions or a fluid admixture thereof can be injected for tissue augmentation or regeneration, including cosmetic tissue augmentation, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a radiopaque hydrolysable crosslinked hydrogel is ultimately formed at the administration location.

During and/or after administration, the compositions of the present disclosure can be imaged using a suitable imaging technique. Typically, the imaging techniques is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to occlude either a vas deferens or fallopian tube with a crosslinked product of the first and second fluid compositions for the control of reproductive health/family planning, a procedure to implant an embolic composition comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions or fluid admixtures thereof may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, injection for obstruction of the vas deferens, injection for obstruction of the fallopian tube, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

In some embodiments, the radiopaque hydrolysable crosslinked hydrogels are formed ex vivo, in which case the radiopaque hydrolysable crosslinked hydrogels may be in any desired form, including a slab, a cylinder, a coating, or a particle. In some embodiments, the radiopaque hydrolysable crosslinked hydrogel is dried and then granulated into particles of suitable size. Granulating may be by any suitable process, for instance by grinding (including cryogrinding), homogenization, crushing, milling, pounding, pressing through a screen, or the like. Sieving or other known techniques can be used to classify and fractionate the particles. Hydrogel particles formed using the above and other techniques may varying widely in size, for example, having an average size ranging from 50 to 950 microns.

In addition to a radiopaque hydrolysable crosslinked hydrogel that is formed ex vivo, hydrogel compositions in accordance with the present disclosure may be provided, which contain additional agents, including therapeutic agents, imaging agents, colorants, tonicity adjusting agents, suspension agents, wetting agents, and pH adjusting agents as described above.

In various embodiments, kits are provided that include one or more delivery devices for delivering such hydrogel compositions to a subject. Such systems may include one or more of the following: a syringe barrel, which may or may not contain a hydrogel composition; a vial, which may or may not contain a hydrogel composition; a needle; a flexible tube (e.g., adapted to fluidly connect the needle to the syringe); and an injectable liquid such as water for injection, normal saline or phosphate buffered saline. Whether supplied in a syringe, vial, or other reservoir, the hydrogel composition may be provided in dry form (e.g., powder form) or in a form that is ready for injection, such as an injectable hydrogel form (e.g., a suspension of hydrogel particles).

FIG. 12 illustrates a syringe 10 providing a reservoir for a hydrogel composition in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a hydrogel composition 15 for injection through the needle 50.

The hydrogel compositions described herein (e.g., a suspension of hydrogel particles, which may also optionally contain additional agents described above) can be used for a number of purposes.

For example, hydrogel compositions can be injected to provide spacing between tissues, hydrogel compositions can be injected (e.g., in the form of blebs) to provide fiducial markers, hydrogel compositions can be injected for tissue augmentation or regeneration, hydrogel compositions can be injected as a filler or replacement for soft tissue, hydrogel compositions can be injected for tissue bulking, hydrogel compositions can be injected for occlusion of lumens, hydrogel compositions can be injected to provide mechanical support for compromised tissue, hydrogel compositions be injected as a scaffold, and/or hydrogel compositions can be injected as a carrier of therapeutic agents in the treatment of diseases and cancers and the repair and regeneration of tissue, among other uses.

During or after administration, the hydrogel compositions of the present disclosure can be imaged using a suitable imaging technique.

As seen from the above, the hydrogel compositions of the present disclosure may be used in a variety of medical procedures, including the following, among others: a procedure to implant a fiducial marker comprising a hydrogel, a procedure to implant a tissue regeneration scaffold comprising a hydrogel, a procedure to implant a tissue support comprising a hydrogel, a procedure to implant a tissue bulking agent comprising a hydrogel, a procedure to occlude a lumen, a procedure to implant a therapeutic-agent-containing depot comprising a hydrogel, a tissue augmentation procedure comprising implanting a hydrogel, a procedure to introduce a hydrogel between a first tissue and a second tissue to space the first tissue from the second tissue, among others.

The hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection to increase coaptation of a bodily sphincter such as an anal sphincter for or a urinary sphincter, injection for vas deferens occlusion, injection for fallopian tube occlusion, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Hydrogel compositions in accordance with the present disclosure include lubricious compositions for medical applications, compositions for therapeutic agent release (e.g., by including one or more therapeutic agents in a matrix of the hydrogel), and implants (which may be formed ex vivo or in vivo) (e.g., compositions for use as tissue markers, compositions that act as spacers to reduce side effects of off-target radiation therapy, cosmetic compositions, etc.).

It should be understood that this disclosure is, in many respects, only illustrative and that changes may be made in details without exceeding the scope of the disclosure. This may include, to the extent that it is appropriate, the use of any of the features of one embodiment being used in other embodiments.

1. A system for forming a hydrogel that comprises (a) a reactive multi-arm polymer that comprises three or more polymer arms linked to a core region, at least three of the three or more polymer arms comprising a cyclic-imidyl ester group, a hydrolysable ester group, an iodinated lactone residue, an amide group and a hydrophilic polymer segment, where the cyclic-imidyl ester group is linked to the iodinated lactone residue through the hydrolysable ester group, the iodinated lactone residue is linked to the hydrophilic polymer segment through the amide group, and the hydrophilic polymer segment is linked to the core region; and (b) a polyamino compound comprising at least two amino (—NH₂) groups, wherein the reactive multi-arm polymer and the polyamino compound react to form a crosslinked hydrogel.

2. The system of claim 1, wherein the hydrophilic polymer segment is selected from a polyalkylene oxide segment, a polyester segment, a polyoxazoline segment, a polydioxanone segment, a polypeptide segment, an acrylic segment, an acrylamide segment, and a polysaccharide segment.

3. The system of claim 1, wherein the iodinated lactone residue comprises at least one iodinated aromatic group.

4. The system of claim 1, wherein the iodinated lactone residue is an iodinated benzolactone residue.

5. The system of claim 4, wherein the iodinated benzolactone residue is selected from an iodinated 1-benzofuran-2-one compound reside, an iodinated 2-benzofuran-1-one compound reside, an iodinated chroman-2-one compound reside, and an iodinated 1-benzoxepin-2-one compound reside.

6. The system of claim 1, wherein the at least three polymer arms comprise a cyclic-imidyl-oxycarbonyl-C₂-C₈-alkylene-carbonyloxy-group linked to the iodinated lactone residue.

7. The system of claim 1, wherein the at least three polymer arms comprise a cyclic-imidyl-oxycarbonyl-1,2-dimethylene-carbonyloxy-end group, a cyclic-imidyl-oxycarbonyl-1,3-trimethylene-carbonyloxy-end group, a cyclic-imidyl-oxycarbonyl-1,4-tetramethylene-carbonyloxy-end group, a cyclic-imidyl-oxycarbonyl-1,5-pentamethylene-carbonyloxy-end group, or a cyclic-imidyl-oxycarbonyl-1,6-hexamethylene-carbonyloxy-end group.

8. The system of claim 1, wherein the core region comprises a polyol residue.

9. The system of claim 1, wherein the polyamino compound comprises a plurality of basic amino acid residues.

10. The system of claim 1, comprising a first composition that comprises the polyamino compound in a first container and a second composition that comprises the reactive multi-arm polymer in a second container.

11. The system of claim 1, further comprising a delivery device.

12. A radiopaque hydrolysable hydrogel produced by covalent crosslinking between the reactive multi-arm polymer and the polyamino compound of claim 1.

13. The radiopaque hydrolysable crosslinked hydrogel of claim 12, wherein the radiopaque hydrolysable hydrogel has a radiopacity that is greater than 100 Hounsfield units (HU).

14. A method of forming a reactive multi-arm polymer that comprises (a) reacting a multi-arm polymer that comprises three or more polymer arms linked to a core region, at least three of the polymer arms comprising the hydrophilic polymer segment and an amino end group, with an iodinated lactone in a ring-opening reaction to form at least three polymer arms that comprise a hydroxy-containing iodinated lactone residue that is linked to the hydrophilic polymer segment through a linkage that comprises an amide group; (b) reacting the reaction product of step (a) with a cyclic anhydride in a ring opening reaction to form at least three polymer arms that comprise carboxyl end groups that a linked to the iodinated lactone residue through a linkage that comprises an ester group; and (c) reacting the reaction product of step (b) with an N-hydroxy cyclic imide compound in an amide coupling reaction to form the reactive multi-arm polymer.

15. The method of claim 14, wherein the cyclic anhydride has a ring that comprises a —C₂-C₈-alkylene-group and wherein the at least three polymer arms comprise a cyclic-imidyl-oxycarbonyl-C₂-C₈-alkylene-carbonyloxy-group.

16. The method of claim 14, wherein the iodinated lactone comprises at least one iodinated aromatic group.

17. The system of claim 14, wherein the iodinated lactone is an iodinated benzolactone.

18. The method of claim 17, wherein the iodinated benzolactone is selected from an iodinated 1-benzofuran-2-one compound, an iodinated 2-benzofuran-1-one compound, an iodinated chroman-2-one compound, and an iodinated 1-benzoxepin-2-one compounds.

19. A method of treatment comprising administering to a subject a mixture that comprises (a) a reactive multi-arm polymer that comprises three or more polymer arms linked to a core region, at least three of the three or more polymer arms comprising a cyclic-imidyl ester group, a hydrolysable ester group, an iodinated lactone residue, an amide group and a hydrophilic polymer segment, where the cyclic-imidyl ester group is linked to the iodinated lactone residue through the hydrolysable ester group, the iodinated lactone residue is linked to the hydrophilic polymer segment through the amide group, and the hydrophilic polymer segment is linked to the core region and (b) a polyamino compound comprising at least two amino (—NH₂) groups, wherein the mixture is administered under conditions such that the polyamino compound and the reactive multi-arm polymer react to form a crosslinked hydrogel after administration.

20. The method of claim 19, wherein the method comprises administering to the subject (a) a first fluid composition that comprises the polyamino compound and the reactive multi-arm polymer, and (b) a second fluid composition that comprises an accelerant that accelerates reaction of the polyamino compound and the reactive multi-arm polymer.
